(19) United States
(12) Patent Application Publication
(10) Pub. No.: US 2010/0311844 A1
(43) Pub. Date: Dec. 9, 2010

(54) Spontaneously Forming Ellipsoidal Phospholipid Unilamellar Vesicles

(76) Inventors: Xiaoyang Qi, Loveland, OH (US); John Katsaras, Deep River (CA); Mu-Ping Nieh, Deep River (CA)

Correspondence Address: BARNES & THORNBURG LLP, 11 SOUTH MERIDIAN, INDIANAPOLIS, IN, 46204, US

(21) Appl. No.: 12/445,707

(22) PCT Filed: Oct. 19, 2007
(86) PCT No.: PCT/US07/81880

§ 371 Date: Aug. 4, 2010

Related U.S. Application Data
(60) Provisional application No. 60/862,312, filed on Oct. 20, 2006.

(51) Int. Cl. A61K 47/42

(52) U.S. Cl. 514/773

(57) ABSTRACT

The instant invention relates generally to compositions and methods useful for the spontaneous formation of liposomes wherein the composition comprises anionic long chain lipids in combination with short chain lipids and a prosaposin-derived protein or polypeptide. The liposomes may be useful for treatment of disease, via administration of the liposome alone or in combination with additional therapeutic agents.

### PRIORITY

This application claims priority to U.S. Provisional Patent Application Ser. No. 60/862,321, entitled “Spontaneously Forming Ellipsoidal Phospholipid Unilamellar Vesicles,” filed Oct. 20, 2006 and U.S. patent application Ser. No. 11/741,323 filed Apr. 27, 2007.

### RELATED APPLICATIONS

This application relates to U.S. Pat. No. 6,872,406 issued Mar. 29, 2005 entitled “Fusogenic Properties of Saposin C and Related Proteins and Peptides for Application to Transmembrane Drug Delivery Systems”; and U.S. patent application Ser. No. 10/801,517, publication No. 2004/0229799 entitled “Saposin C-DOPS: A Novel Anti-Tumor Agent” all of which are incorporated in their entirety herein by reference.

### FIELD OF THE INVENTION

This present invention relates to a phospholipids composition for targeted drug delivery and improved therapeutics. A pharmaceutical agent is contained within a phospholipids membrane and delivery is facilitated by a membrane fusion protein. More specifically, the pharmaceutical agent is contained within a liposome, and delivery is facilitated using Saposin C, which is in association with the liposome.

### BACKGROUND

Liposomes are microscopic vesicles that have a central aqueous cavity surrounded by a lipid membrane formed by concentric bilayer(s). The liposomes can be unilamellar (having only one lipid bilayer), oligolamellar or multilamellar (having multiple bilayers). Their structure allows them to incorporate either hydrophilic substances in the aqueous interior or hydrophobic substances in the lipid membrane.

As vehicles for the administration of drugs, liposomes have, in theory, numerous advantages. As well as being composed of non-toxic components, generally non-immunogenic, non-irritant and biodegradable, they should be capable of encapsulating, retaining, transporting and releasing a large variety of therapeutic agents to target organs, thereby reducing adverse side effects. Liposomes can form the basis for sustained drug release and delivery to specific cell types, or parts of the body. The therapeutic use of liposomes also includes the delivery of drugs which are normally toxic in free form.

Generally, liposomes are formed by subjecting a mixture of phospholipids to a mechanical force. For example, a wide variety of methods are currently used in the preparation of liposome compositions. These include, for example, solvent dialysis, French press, extrusion (with or without freeze-thaw), reverse phase evaporation, simple freeze-thaw, sonication, chelate dialysis, homogenization, solvent infusion, microemulsification, spontaneous formation, solvent vaporization, French pressure cell technique, controlled detergent dialysis, and others. See, e.g., Madden et al., Chemistry and Physics of Lipids, 1990. Liposomes may also be formed by various processes which require shaking or vortexing.

Problems associated with liposomes include colloidal instability, difficulty in scale-up sterilization, and variability between batches in manufacturing. Liposome preparation and manufacturing typically involves removal of organic solvents followed by extrusion or homogenization. These processes may expose liposomal components to extreme conditions such as elevated pressures, elevated temperatures and high shear conditions which can degrade lipids and other molecules incorporated into the liposomes.

Liposome preparations are often characterized by very heterogeneous distributions of sizes and number of bilayers. Conditions optimized on a small scale normally do not scale up well and preparation of large-scale batches is cumbersome and labor intensive.

Another problem for liposome applications is colloidal stability. Liposomes in suspension can aggregate and fuse upon storage, heating and addition of various additives. Because of these stability problems, liposomes are often lyophilized. Lyophilization is costly and time consuming. Upon reconstitution, size distributions often increase and encapsulated materials may leak out from the liposomes.

Most, if not all, known liposome suspensions are not thermodynamically stable. Instead, the liposomes in known suspensions are kinetically trapped into higher energy states by the energy used in their formation. Energy may be provided as heat, sonication, extrusion, or homogenization. Since every high-energy state tries to lower its free energy, known liposome formulations experience problems with aggregation, fusion, sedimentation and leakage of liposome associated material. A thermodynamically stable liposome formulation which could avoid some of these problems is therefore desirable.

It is therefore desirable to develop new methods and materials which address these problems with current liposome formulations.

### BRIEF SUMMARY OF THE INVENTION

The present invention relates generally to a composition for forming a population of liposomes useful for treatment of disease or delivery of active agents to an individual comprising a) at least one long-chain anionic phospholipid; b) at least one short-chain phospholipid; c) and a prosaposin-derived protein or polypeptide; wherein the liposome is spontaneously formed upon addition of an aqueous solution.

In one embodiment, the anionic phospholipid may be selected from the group consisting of dioleoylphosphatidylserine (DOPS), dioleoylphosphatidylglycerol (DOPG), dioleoylphosphatidylinositol (DOPI) and dioleoylphosphatidic acid (DOPA).

In a further embodiment, the short-chain phospholipid may be selected from the group consisting of a phosphatidylserine, a phosphatidylcholine, a phosphatidylglycerol, a phosphatidylinositol, a phosphatidic acid, and a phosphatidylethanolamine.

The compositions may further comprise a population of liposomes has a monomodal, bimodal, or trimodal unilamellar vesicles size distribution or comprise oblate and tri-axial ellipsoidal unilamellar vesicles.

In a further embodiment, the composition the prosaposin-derived protein is one or more selected from the group consisting of saposin C, H1, H2, H3, H4, H5 or mixtures thereof.

In a yet further embodiment, the composition for forming a liposome comprises DOPS, DPPC, DHPC and a prosaposin-derived protein or polypeptide selected from the group consisting of saposin C, H1 peptide, H2 peptide, H3 peptide, H4 peptide, H5 peptide or mixtures thereof.

In a yet further embodiment, the composition for forming a liposome comprises DOPS, DHPS and a prosaposin-derived protein or polypeptide selected from the group consisting of saposin C, H1 peptide, H2 peptide, H3 peptide, H4 peptide, H5 peptide or mixtures thereof.

In a yet further embodiment, the composition for forming a liposome comprises DOPS, DHPS, DPPC, DHPC and a prosaposin-derived protein or polypeptide selected from the group consisting of saposin C, H1 peptide, H2 peptide, H3 peptide, H4 peptide, H5 peptide or mixtures thereof.

In a yet further embodiment, the composition further comprises a pharmaceutically active agent.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

FIG. 1: Amino acid sequence of human Saposin C and its functional domains.

FIG. 2: SANS data of 0.1% DOPS/DPPC/DHPC.

FIG. 3: Modified Guinier plot for the 0.1 wt. % DOPS/DPPC/DHPC mixture.

FIG. 4: Representative TEM images of a DOPS/DPPC/DHPC mixture (A and B), and H1-doped (C and D), and H2-doped (E and F) mixtures.

FIG. 5: The proposed model for a unilamellar, tri-axial ellipsoidal bilayered vesicle.

### DETAILED DESCRIPTION OF THE INVENTION

### Abbreviations

Sap C, Saposin C; DOPG, Dioleoylphosphatidylglycerol; DOPS, Dioleoylphosphatidylserine; PC, Phosphoatidyecholines; PG, Phosphatidylglycerol; PS, Phosphatidylserine; DMPC, Dimyristylphosphatidylcholine Definitions

Before the present compositions and methods are described, it is to be understood that this invention is not limited to the specific methodology, devices, and formulations as such may, of course, vary. It is also to be understood that the terminology used herein is for the purpose of describing particular embodiments only, and is not intended to limit the scope of the present invention which will be limited only by the appended claims, and equivalents thereof.

As used herein and in the appended claims, the singular forms “a”, “and”, and “the” include plural referents unless the context clearly dictates otherwise. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, exemplary methods, devices and materials are now described.

The terms “administered” and “administration” refer generally to the administration to a patient of a biocompatible material, including, for example, lipid and/or vesicle compositions and flush agents. Accordingly, “administered” and “administration” refer, for example, to the injection into a blood vessel of lipid and/or vesicle compositions and/or flush agents. The terms “administered” and “administration” can also refer to the delivery of lipid and/or vesicle compositions and/or flush agents to a region of interest.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

The term “amphipathic lipid” means a molecule that has a hydrophilic “head” group and hydrophobic “tail” group and has membrane-forming capability.

By “analogs” is meant substitutions or alterations in the amino acid sequences of the peptides of the invention, which substitutions or alterations do not adversely affect the fusogenic properties of the peptides. Thus, an analog might comprise a peptide having a substantially identical amino acid sequence to a peptide provided herein and in which one or more amino acid residues have been conservatively substituted with chemically similar amino acids. Examples of conservative substitutions include the substitution of a non-polar (hydrophobic) residue such as isoleucine, valine, leucine or methionine for another. Likewise, the present invention contemplates the substitution of one polar (hydrophilic) residue such as between arginine and lysine, between glutamine and asparagine, and between glycine and serine. Additionally, the substitution of a basic residue such as lysine, arginine or histidine for another or the substitution of one acidic residue such as aspartic acid or glutamic acid for another is also contemplated.

As used herein, the terms “anionic phospholipid membrane” and “anionic liposome” refer to a phospholipid membrane or liposome that contains lipid components and has an overall negative charge at physiological pH.

“Anionic phospholipids” means phospholipids having negative charge, including phosphate, sulphate and glycerol-based lipids.

“Bioactive agent” refers to a substance which may be used in connection with an application that is therapeutic or diagnostic in nature, such as in methods for diagnosing the presence or absence of a disease in a patient and/or in methods for the treatment of disease in a patient. As used herein, “bioactive agent” refers also to substances which are capable of exerting a biological effect in vitro and/or in vivo. The bioactive agents may be neutral or positively or negatively charged. Examples of suitable bioactive agents include diagnostic agents, pharmaceuticals, drugs, synthetic organic molecules, proteins, peptides, vitamins, steroids and genetic material, including nucleosides, nucleotides and polynucleotides.

The term “contained (with)in” refers to a pharmaceutical agent being enveloped within a phospholipid membrane, such that the pharmaceutical agent is protected from the outside environment. This term may be used interchangeably with “encapsulated.”

A “deletion,” as the term is used herein, refers to a change in the amino acid or nucleotide sequence that results in the absence of one or more amino acid residues or nucleotides.

The term “derivative,” as used herein, refers to the chemical modification of a polypeptide sequence, or a polynucleotide sequence. Chemical modifications of a polynucleotide sequence can include, for example, replacement of hydrogen by an alkyl, acyl, or amino group. A derivative polynucleotide encodes a polypeptide which retains at least one biological function of the natural molecule. A derivative polypeptide is one modified, for instance by glycosylation, or any other process which retains at least one biological function of the polypeptide from which it was derived.

The term “fusogenic protein or polypeptide” as used herein refers to a protein or peptide that when added to two separate bilayer membranes can bring about their fusion into a single membrane. The fusogenic protein forces the cell or model membranes into close contact and causes them to fuse.

The words “insertion” or “addition,” as used herein, refer to changes in an amino acid or nucleotide sequence resulting in the addition of one or more amino acid residues or nucleotides, respectively, to the sequence found in the naturally occurring molecule.

The terms “lipid” and “phospholipid” are used interchangeably and to refer to structures containing lipids, phospholipids, or derivatives thereof comprising a variety of different structural arrangements which lipids are known to adopt in aqueous suspension. These structures include, but are not limited to, lipid bilayer vesicles, micelles, liposomes, emulsions, vesicles, lipid ribbons or sheets. The lipids may be used alone or in any combination which one skilled in the art would appreciate to provide the characteristics desired for a particular application. In addition, the technical aspects of lipid constructs and liposome formation are well known in the art and any of the methods commonly practiced in the field may be used for the present invention.

“Lipid composition” refers to a composition which comprises a lipid compound, typically in an aqueous medium. Exemplary lipid compositions include suspensions, emulsions and vesicle compositions. “Lipid formulation” refers to a lipid composition which also comprises a bioactive agent.

“Liposome” refers to a generally spherical cluster or aggregate of amphipathic compounds, including lipid compounds, typically in the form of one or more concentric layers, for example, bilayers. They may also be referred to herein as lipid vesicles.

The term “long-chain lipid” refers to lipids having a carbon chain length of about 13, 14, 15, 16, 17, 18, 19, 20, 21, 22, 23 or 24. In one embodiment, the chain length is selected from a chain length of 18, 19, or 20. Examples of lipids that may be used with the present invention are available on the website www.avantilipids.com. Representative examples of long chain lipids that may be used with the present invention include, but are not limited to the following lipids:

14:0 PS 1,2-Dimyristoyl-sn-Glycero-3-[Phospho-L-Serine] (Sodium Salt) (DMPS); 16:0 PS 1,2-Dipalmitoyl-sn-Glycero-3-[Phospho-L-Serine] (Sodium Salt) (DPPS); 17:0 PS 1,2-Diheptadecanoyl-sn-Glycero-3-[Phospho-L-Serine] (Sodium Salt); 18:0 PS 1,2-Distearoyl-sn-Glycero-3-[Phospho-L-Serine] (Sodium Salt) (DSPS); 18:1 PS 1,2-Dioleoyl-sn-Glycero-3-[Phospho-L-Serine] (Sodium Salt) (DOPS); 18:2 PS 1,2-Dilinoleoyl-sn-Glycero-3-[Phospho-L-Serine] (Sodium Salt); 20:4 PS 1,2-Diarachidonoyl-sn-Glycero-3-[Phospho-L-Serine] (Sodium Salt); 22:6 PS 1,2-Didocosahexaenoyl-sn-Glycero-3-[Phospho-L-Serine] (Sodium Salt); 16:0-18:1 PS 1-Palmitoyl-2-Oleoyl-sn-Glycero-3-[Phospho-L-Serine] (Sodium Salt) (POPS); 16:0-18:2 PS 1-Palmitoyl-2-Linoleoyl-sn-Glycero-3-[Phospho-L-Serine] (Sodium Salt); 16:0-22:6 PS 1-Palmitoyl-2-Docosahexaenoyl-sn-Glycero-3-[Phospho-L-Serine](Sodium Salt); 18:0-18:1 PS 1-Stearoyl-2-Oleoyl-sn-Glycero-3-[Phospho-L-Serine] (Sodium Salt); 18:0-18:2 PS 1-Stearoyl-2-Linoleoyl-sn-Glycero-3-[Phospho-L-Serine] (Sodium Salt); 18:0-20:4 PS 1-Stearoyl-2-Arachidonoyl-sn-Glycero-3-[Phospho-L-Serine] (Sodium Salt); 18:0-22:6 PS 1-Stearoyl-2-Docosahexaenoyl-sn-Glycero-3-[Phospho-L-Serine] (Sodium Salt); 16:0 PC 1,2-Dipalmitoyl-sn-Glycero-3-Phosphocholine (DPPC); 17:0 PC 1,2-Diheptadecanoyl-sn-Glycero-3-Phosphocholine; 18:0 PC 1,2-Distearoyl-sn-Glycero-3-Phosphocholine (DSPC); 16:1 PC (Cis) 1,2-Dipalmitoleoyl-sn-Glycero-3-Phosphocholine; 16:1 Trans PC 1,2-Dipalmitelaidoyl-sn-Glycero-3-Phosphocholine; 18:1 PC Delta6 (cis) 1,2-Dipetroselinoyl-sn-Glycero-3-Phosphocholine; 18:2 PC (cis) 1,2-Dilinoleoyl-sn-Glycero-3-Phosphocholine; 18:3 PC (cis) 1,2-Dilinolenoyl-sn-Glycero-3-Phosphocholine; 20:1 PC (cis) 1,2-Dieicosenoyl-sn-Glycero-3-Phosphocholine; 22:1 PC (cis) 1,2-Dierucoyl-sn-Glycero-3-Phosphocholine; 22:0 PC 1,2-Dibehenoyl-sn-Glycero-3-Phosphocholine; 24:1 PC (cis) 1,2-Dinervonoyl-sn-Glycero-3-Phosphocholine; 16:0-18:0 PC 1-Palmitoyl-2-Stearoyl-sn-Glycero-3-Phosphocholine; 16:0-18:1 PC 1-Palmitoyl-2-Oleoyl-sn-Glycero-3-Phosphocholine; 16:0-18:2 PC 1-Palmitoyl-2-Linoleoyl-sn-Glycero-3-Phosphocholine; 18:0-18:1 PC 1-Stearoyl-2-Oleoyl-sn-Glycero-3-Phosphocholine; 18:0-18:2 PC 1-Stearoyl-2-Linoleoyl-sn-Glycero-3-Phosphocholine; 18:1-18:0 PC 1-Oleoyl-2-Stearoyl-sn-Glycero-3-Phosphocholine; 18:1-16:0 PC 1-Oleoyl-2-Palmitoyl-sn-Glycero-3-Phosphocholine; 18:0-20:4 PC 1-Stearoyl-2-Arachidonyl-sn-Glycero-3-Phosphocholine; 16:0-18:1 PG 1-Palmitoyl-2-Oleoyl-sn-Glycero-3-[Phospho-rac-(1-glycerol)] (Sodium Salt) (POPG); 18:1 PG 1,2-Dioleoyl-sn-Glycero-3-[Phospho-rac-(1-glycerol)] (Sodium Salt) (DOPG); 18:1 PA 1,2-Dioleoyl-sn-Glycero-3-Phosphate (Monosodium Salt) (DOPA); 18:1 PI 1,2-Dioleoyl-sn-Glycero-3-Phosphoinositol (Ammonium Salt); 16:0 (D31)-18:1 PI 1-Palmitoyl(D31)-2-Oleoyl-sn-Glycero-3-Phosphoinositol (Ammonium Salt); 18:1 PE 1,2-Dioleoyl-sn-Glycero-3-Phosphoethanolamine (DOPE); 18:2 PE 1,2-Dilinoleoyl-sn-Glycero-3-Phosphoethanolamine.

The phrases “nucleic acid” or “nucleic acid sequence,” as used herein, refer to a nucleotide, oligonucleotide, polynucleotide, or any fragment thereof. A “nucleic acid” refers to a string of at least two base-sugar-phosphate combinations. (A polynucleotide is distinguished from an oligonucleotide by containing more than 120 monomeric units.) Nucleotides are the monomeric units of nucleic acid polymers. The term includes deoxyribonucleic acid (DNA) and ribonucleic acid (RNA) in the form of an oligonucleotide messenger RNA, anti-sense, plasmid DNA, parts of a plasmid DNA or genetic material derived from a virus. Anti-sense is a polynucleotide that interferes with the function of DNA and/or RNA. The term nucleic acid refers to a string of at least two base-sugar-phosphate combinations. Natural nucleic acids have a phosphate backbone, artificial nucleic acids may contain other types of backbones, but contain the same bases. Nucleotides are the monomeric units of nucleic acid polymers. The term includes deoxyribonucleic acid (DNA) and ribonucleic acid (RNA). RNA may be in the form of a tRNA (transfer RNA), siRNA (short interfering ribonucleic acid), snRNA (small nuclear RNA), rRNA (ribosomal RNA), mRNA (messenger RNA), anti-sense RNA, and ribozymes. DNA may be in form plasmid DNA, viral DNA, linear DNA, or chromosomal DNA or derivatives of these groups. In addition these forms of DNA and RNA may be single, double, triple, or quadruple stranded. The term also includes PNAs (peptide nucleic acids), siNA (short interfering nucleic acid), phosphorothioates, and other variants of the phosphate backbone of native nucleic acids.

As used herein, the term “nucleotide-based pharmaceutical agent” or “nucleotide-based drug” refer to a pharmaceutical agent or drug comprising a nucleotide, an oligonucleotide or a nucleic acid.

“Patient” or “subject” or “individual” refers to animals, including mammals, preferably humans.

As used herein, “pharmaceutical agent” or “active agent” or “drug” refers to any chemical or biological material, compound, or composition capable of inducing a desired therapeutic effect when properly administered to a patient. Some drugs are sold in an inactive form that is converted in vivo into a metabolite with pharmaceutical activity. For purposes of the present invention, the terms “pharmaceutical agent” “active agent” and “drug” encompass both the inactive drug and the active metabolite.

The phrase “pharmaceutically or therapeutically effective dose or amount” refers to a dosage level sufficient to induce a desired biological result. That result may be the delivery of a pharmaceutical agent, alleviation of the signs, symptoms or causes of a disease or any other desired alteration of a biological system and the precise amount of the active depends on the physical condition of the patient, progression of the illness being treated etc.

As used herein, the term “peptide analog” refers to a peptide which differs in amino acid sequence from the native peptide only by conservative amino acid substitutions, for example, substitution of Leu for Val, or Arg for Lys, etc., or by one or more non-conservative amino acid substitutions, deletions, or insertions located at positions which do not destroy the biological activity of the peptide (in this case, the fusogenic property of the peptide). A peptide analog, as used herein, may also include, as part or all of its sequence, one or more amino acid analogues, molecules which mimic the structure of amino acids, and/or natural amino acids found in molecules other than peptide or peptide analogues.

As used herein, the term “prosaposin-derived proteins and polypeptides” includes but is not limited to naturally occurring saposins A, B, C and D. The phrase term further includes synthetic saposin-derived proteins and peptides and peptide analogs having similar or substantially similar biological activity, such as, for example, membrane interaction for organizing the membrane structures, lipid binding and transfer functions, lipid presentation, membrane restructuring, membrane anchoring, etc. The saposin C and polypeptides derived therefrom may be used in one embodiment of the invention. The term further includes fragments such as the H1, H2, H3, H4 or H5 fragments described herein and/or known in the art and biologically active equivalents thereto.

The term “short chain lipid” refers to lipids having a carbon chain length of 4, 5, 6, 7, 8, 9, 10, 11 or 12. In one embodiment, the carbon chain length is 6, 7, 8 9 or 10. In one embodiment, the carbon chain length is 6, 7 or 8. Examples of negative short chain lipids are available at the website www.avantilipids.com. Examples of short chain lipids that may be used with the present invention include, but are not limited to, the following:

06:0 PS (DHPS) 1,2-Dihexanoyl-sn-Glycero-3-[Phospho-L-Serine] (Sodium Salt); 08:0 PS 1,2-Dioctanoyl-sn-Glycero-3-[Phospho-L-Serine] (Sodium Salt); 03:0 PC 1,2-Dipropionoyl-sn-Glycero-3-Phosphocholine; 04:0 PC 1,2-Dibutyroyl-sn-Glycero-3-Phosphocholine; 05:0 PC 1,2-Divaleroyl-sn-Glycero-3-Phosphocholine; 06:0 PC (DHPC) 1,2-Dihexanoyl-sn-Glycero-3-Phosphocholine; 07:0 PC 1,2-Diheptanoyl-sn-Glycero-3-Phosphocholine; 08:0 PC 1,2-Dioctanoyl-sn-Glycero-3-Phosphocholine; 09:0 PC 1,2-Dinonanoyl-sn-Glycero-3-Phosphocholine; 06:0 PG 1,2-Dihexanoyl-sn-Glycero-3-[Phospho-rac-(1-glycerol)] (Sodium Salt); 08:0 PG 1,2-Dioctanoyl-sn-Glycero-3-[Phospho-rac-(1-glycerol)] (Sodium Salt); 06:0 PA 1,2-Dihexanoyl-sn-Glycero-3-Phosphate (Monosodium Salt); 08:0 PA 1,2-Dioctanoyl-sn-Glycero-3-Phosphate (Monosodium Salt); 06:0 PE 1,2-Dihexanoyl-sn-Glycero-3-Phosphoethanolamine; 08:0 PE 1,2-Dioctanoyl-sn-Glycero-3-Phosphoethanolamine.

As used herein, the term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule”, refers to any nucleic acid molecule capable of inhibiting or down regulating gene expression or viral replication, for example by mediating RNA interference “RNAi” or gene silencing in a sequence-specific manner. Within exemplary embodiments, the siNA is a double-stranded polynucleotide molecule comprising self-complementary sense and antisense regions, wherein the antisense region comprises a nucleotide sequence that is complementary to a nucleotide sequence in a target nucleic acid molecule for down regulating expression, or a portion thereof, and the sense region comprises a nucleotide sequence corresponding to (i.e., which is substantially identical in sequence to) the target nucleic acid sequence or portion thereof. “siNA” means a small interfering nucleic acid, for example a siRNA, that is a short-length double-stranded nucleic acid (or optionally a longer precursor thereof), and which is not unacceptably toxic in target cells. The length of useful siNAs within the invention will in certain embodiments be optimized at a length of approximately 21 to 23 by long. However, there is no particular limitation in the length of useful siNAs, including siRNAs. For example, siNAs can initially be presented to cells in a precursor form that is substantially different than a final or processed form of the siNA that will exist and exert gene silencing activity upon delivery, or after delivery, to the target cell. Precursor forms of siNAs may, for example, include precursor sequence elements that are processed, degraded, altered, or cleaved at or following the time of delivery to yield a siNA that is active within the cell to mediate gene silencing. Thus, in certain embodiments, useful siNAs within the invention will have a precursor length, for example, of approximately 100-200 base pairs, 50-100 base pairs, or less than about 50 base pairs, which will yield an active, processed siNA within the target cell. In other embodiments, a useful siNA or siNA precursor will be approximately 10 to 49 bp, 15 to 35 bp, or about 21 to 30 by in length.

As used herein, the term “spontaneously formed” is intended to encompass that meaning known in the art, wherein the formation of the liposome requires the application of minimal or no mechanical force to the mixture of phospholipids, though it is to be understood that the application of mechanical force, such as via vortexing or mixing, may be applied to facilitate formation of the liposome composition.

The terms “stable” or “stabilized”, as used herein, means that the vesicles may be substantially resistant to degradation, including, for example, loss of vesicle structure or encapsulated gas or gaseous precursor, for a useful period of time. Typically, the vesicles employed in the present invention have a desirable shelf life, often retaining at least about 90% by volume of its original structure for a period of at least about two to three weeks under normal ambient conditions. In preferred form, the vesicles are desirably stable for a period of time of at least about 1 month, more preferably at least about 2 months, even more preferably at least about 6 months, still more preferably about eighteen months, and yet more preferably up to about 3 years. The vesicles described herein, including gas and gaseous precursor filled vesicles, may also be stable even under adverse conditions, such as temperatures and pressures which are above or below those experienced under normal ambient conditions.

“Vesicle” refers to a spherical entity which is generally characterized by the presence of one or more walls or membranes which form one or more internal voids. Vesicles may be formulated, for example, from lipids, including the various lipids described herein, proteinaceous materials, polymeric materials, including natural, synthetic and semi-synthetic polymers, or surfactants. Preferred vesicles are those which comprise walls or membranes formulated from lipids. In these preferred vesicles, the lipids may be in the form of a monolayer or bilayer, and the mono- or bilayer lipids may be used to form one or more mono- or bilayers. In the case of more than one mono- or bilayer, the mono- or bilayers may be concentric. Lipids may be used to form a unilamellar vesicle (comprised of one monolayer or bilayer), an oligolamellar vesicle (comprised of about two or about three monolayers or bilayers) or a multilamellar vesicle (comprised of more than about three monolayers or bilayers). Similarly, the vesicles prepared from proteins or polymers may comprise one or more concentric walls or membranes. The walls or membranes of vesicles prepared from proteins or polymers may be substantially solid (uniform), or they may be porous or semi-porous. The vesicles described herein include such entities commonly referred to as, for example, liposomes, micelles, bubbles, microbubbles, microspheres, lipid-, polymer- protein- and/or surfactant-coated bubbles, microbubbles and/or microspheres, microballoons, aerogels, clathrate bound vesicles, and the like. The internal void of the vesicles may be filled with a liquid (including, for example, an aqueous liquid), a gas, a gaseous precursor, and/or a solid or solute material, including, for example, a targeting ligand and/or a bioactive agent, as desired.

### DESCRIPTION

The subject of the present disclosure relates generally to unilamellar phospholipid vesicles such as liposomes that may be spontaneously formed upon the combining of an aqueous solution with selected phospholipids. The vesicles are easily formed, stable, non-leaky (i.e., do not release their contents) and cover a wide size range. Prosaposin-derived proteins, such as Saposin C, and/or the H1 and H2 regions of SapC may be incorporated into the phospholipid vesicles. The phospholipid vesicles, or liposomes, described herein are useful for treatment of disease. The liposomes containing lipids and the prosaposin-derived protein or polypeptide may be used as therapeutic agents in the absence of additional pharmaceutical agents, such as in the treatment of disease states such as cancer, or may further be used to deliver and administer pharmaceutically active agents, particularly where delivery across a biological membrane is advantageous.

In brief, the liposomes of the instant invention generally comprise one or more long-chain, anionic lipids and one or more prosaposin-derived protein or polypeptide, wherein the unique combination of lipids allows for the spontaneous formation of the liposomes.

In one embodiment, the liposomes are comprised of a combination of one or more anionic long chain lipids, one or more short chain phospholipids, and one or more prosaposin-derived protein or polypeptides or analogues or derivatives thereof.

In a yet further embodiment, the liposomes of the instant invention may further comprise one or more pharmaceutical agent for the delivery of that agent to an individual in need of such treatment.

In general, the method of making the liposomes described herein comprise the steps of providing one or more dry phospholipids and a prosaposin-derived protein or polypeptide or analogue or derivative thereof; adding an aqueous solution to form a mixture; allowing the mixture to form liposomes.

In an alternative embodiment, the method may comprise the steps of providing one or more dry phospholipids and a prosaposin-derived protein or polypeptide; adding an organic solvent for form a first mixture; freeze-drying the first mixture; adding an aqueous solution to the first mixture to form a second mixture; allowing the second mixture to form liposomes. The one or more dry phospholipids may comprise at least one anionic long-chain phospholipid and/or at least one short chain phospholipid. The prosaposin-derived protein or polypeptide may be Saposin C or a fragment such as H1, H2, H3, H4 or H5.

In another embodiment of the present invention, the pH of the protein-lipid composition is acidic. In another embodiment of the present invention, the pH of the composition is between about 6.8 and 2. In another embodiment of the present invention, the pH of the composition is between about 5.5 and 2. In another embodiment, the pH is between about 5.5 and about 3.5.

In another embodiment, the protein and lipid composition in dry form is treated with an acid. In one embodiment, the acid is an acidic buffer or organic acid. In another embodiment, the acid is added at a level sufficient to protonate at least a portion of the protein, wherein the composition has a pH of from about 5.5 and about 2. In another embodiment, the acid is added at a level sufficient to substantially protonate the protein, wherein the composition has a pH of from about 5.5 and about 2.

In a further embodiment, the pH of the protein and lipid composition dry powder that has been treated with an acid sufficient to protonate at least a portion of the protein is then substantially neutralized. In one embodiment, the pH is neutralized with a neutral pH buffer. In one embodiment, the pH is neutralized with a neutral pH buffer sufficiently to control the size of the resulting liposome. In another embodiment, the pH is neutralized with a neutral pH buffer sufficiently to control the size of the resulting liposome to provide for liposomes having mean diameters of about 200 nanometers. In another embodiment, the liposomes have a mean diameter of between 50 and 350 nanometers. In another embodiment, the liposomes have a mean diameter of between 150 and 250 nanometers. In another embodiment, the buffer is added to the composition to provide a final composition pH of from about 5 to about 14, preferably from about 7 to 14, more preferably from about 7 to about 12, more preferably from about 7 to about 10, and even more preferably from about 8 to about 10.

The liposomes and associated methods of the instant invention are uniquely characterized in that the liposomes may be spontaneously formed upon the addition of an aqueous solution, such that application of a mechanical force is not required. Further, the resulting liposomal population has an extended shelf life on the order of years or more. As such, in some embodiments of the instant invention, one of skill in the art may readily provide liposomal based delivery systems or treatments with reduced financial investment in reagents and equipment, and reduces exposure to toxic reagents and costs associated with disposal of such reagents.

### Fusogenic Proteins or Polypeptides

Saposins, a family of small (˜80 amino acids) heat stable glycoproteins, are essential for the in vivo hydrolytic activity of several lysosomal enzymes in the catabolic pathway of glycosphingolipids (see Grabowski, G. A., Gatt, S., and Horowitz, M. (1990) Crit. Rev. Biochem. Mol. Biol. 25, 385-414; Furst, W., and Sandhoff, K., (1992) Biochim. Biophys. Acta 1126, 1-16; Kishimoto, Y., Kiraiwa, M., and O'Brien, J. S. (1992) J. Lipid. Res. 33, 1255-1267). Four members of the saposin family, A, B, C, and D, are proteolytically hydrolyzed from a single precursor protein, prosaposin (see Fujibayashi, S., Kao, F. T., Hones, C., Morse, H., Law, M., and Wenger, D. A. (1985) Am. J. Hum. Genet. 37, 741-748; O'Brien, J. S., Kretz, K. A., Dewji, N., Wenger, D. A., Esch, F., and Fluharty, A. L. (1988) Science 241, 1098-1101; Rorman, E. G., and Grabowski, G. A. (1989) Genomics 5, 486-492; Nakano, T., Sandhoff, K., Stumper, J., Christomanou, H., and Suzuki, K. (1989) J. Biochem. (Tokyo) 105, 152-154; Reiner, O., Dagan, O., and Horowitz, M. (1989) J. Mol. Neurosci. 1, 225-233). The complete amino acid sequences for saposins A, B, C and D have been reported as well as the genomic organization and cDNA sequence of prosaposin (see Fujibayashi, S., Kao, F. T., Jones, C., Morse, H., Law, M., and Wenger, D. A. (1985) Am. J. Hum. Genet. 37, 741-748; O'Brien, J. S., Kretz, K. A., Dewji, N., Wenger, D. A., Esch, F., and Fluharty, A. L. (1988) Science 241, 1098-1101; Rorman, E. G., and Grabowski, G. A. (1989) Genomics 5, 486-492). A complete deficiency of prosaposin with mutation in the initiation codon causes the storage of multiple glycosphingolipid substrates resembling a combined lysosomal hydrolase deficiency (see Schnabel, D., Schroder, M., Furst, W., Klien, A., Hurwitz, R., Zenk, T., Weber, J., Harzer, K., Paton, B. C., Poulos, A., Suzuki, K., and Sandhoff, K. (1992) J. Biol. Chem. 267, 3312-3315).

Saposins are defined as sphingolipid activator proteins or coenzymes. Structurally, saposins A, B, C, and D have approximately 50-60% similarity including six strictly conserved cysteine residues (see Furst, W., and Sandhoff, K., (1992) Biochim. Biophys. Acta 1126, 1-16) that form three intradomain disulfide bridges whose placements are identical (see Vaccaro, A. M., Salvioli, R., Barca, A., Tatti, M., Ciaffoni, F., Maras, B., Siciliano, R., Zappacosta, F., Amoresano, A., and Pucci, P. (1995) J. Biol. Chem. 270, 9953-9960). All saposins contain one glycosylation site with conserved placement in the N-terminal sequence half, but glycosylation is not essential to their activities (see Qi. X., and Grabowski, G. A. (1998) Biochemistry 37, 11544-11554; Vaccaro, A. M., Ciaffoni, F., Tatti, M., Salvioli, R., Barca, A., Tognozzi, D., and Scerch, C. (1995) J. Biol. Chem. 270, 30576-30580). In addition, saposin A has a second glycosylation site in C-terminal half.

All saposins and saposin-like proteins and domains contain a “saposin fold” when in solution. This fold is a multiple α-helical bundle motif, characterized by a three conserved disulfide structure and several amphipathic polypeptides. Despite this shared saposin-fold structure in solution, saposins and saposin-like proteins have diverse in vivo biological functions in the enhancement of lysosomal sphingolipid (SL) and glycosphingolipid (GSL) degradation by specific hydrolases. Because of these roles, the saposins occupy a central position in the control of lysosomal sphingolipid and glycosphingolipid metabolisms.

In the absence of this function, glucosylceramide accumulates in the brain leading to Gaucher disease. (see Pàmpols, T.; Pineda, M.; Girós, M. L.; Ferrer, I.; Cusi, V.; Chabás, A.; Sammarti, F. X.; Vanier, M. T.; Christomanou, H. Acta Neuropatol. 1999, 97, 91-97) Another disease resulting from the accumulation of glycosphingolipids (GSL) is metachromatic leukodystrophy, which may also be caused by deficiencies of lysosomal enzyme and saposin activators. (see Zhang, X. L.; Rafi, M. A.; DeGala, G.; Wenger, D. A. Proc Natl Acad Sci USA 1990, 87, 1426-1430; Schnabel, D.; Schroder, M.; Sandhoff, K. FEBS Lett 1991, 284, 57-59) As well as its specific role in enzymatic activation, SapC is also capable of neuritogenic activity, inter-membrane transport of gangliosides and GSL, lipid antigen presentation and membrane-fusion induction activities. It should be noted that the intravenous administration of SapC bound to PS ULV, has also been used to demonstrate the ability to transport fluorescent labeled phospholipid into the central nerve system. It therefore seems, that the combined SapC-PS complex may lend itself to a new drug and gene delivery system specific to the treatment of neurological and brain diseases.

Suitable fusogenic proteins and polypeptides for use in this invention include, but are not limited to, proteins of the saposin family, for example, saposin C. Also included are homologues of saposin C, wherein the homologue possesses at least 80% sequence homology, or at least 90% sequence homology, such that the fragment possesses similar or substantially similar biological activity. Due to degeneracy of the genetic code which encodes for saposin C, it will be readily understood by one of ordinary skill in the art that 100% sequence homology is not essential to operation of the instant invention.

Examples of peptides or peptide analogues include:

(SEQ. ID. No. 1)
Ser-Asp-Val-Tyr-Cys-Glu-Val-Cys-Glu-Phe-Leu-Val-
Lys-Glu-Val-Thr-Lys-Leu-Ile-Asp-Asn-Asn-Lys-Thr-
Glu-Lys-Glu-Ile-Leu-Asp-Ala-Phe-Asp-Lys-Met-Cys-
Ser-Lys-Leu-Pro;
(SEQ. ID. No. 2)
Val-Tyr-Cys-Glu-Val-Cys-Glu-Phe-Leu-Val-Lys-Glu-
Val-Thr-Lys-Leu-Ile-Asp-Asn-Asn-Lys-Thr-Glu-Lys-
Glu-Ile-Leu-Asp-Ala-Phe-Asp-Lys-Met-Cys-Ser-Lys-
Leu-Pro,

and derivatives, analogues, homologues, fragments and mixtures thereof.

Also included are polypeptides of the formula:

h-u-Cys-Glu-h-Cys-Glu-h-h-h-Lys-Glu-h-u-Lys-h-h-
Asp-Asn-Asn-Lys-u-Glu-Lys-Glu-h-h-Asp-h-h-Asp-
Lys-h-Cys-u-Lys-h-h,

where h=hydrophobic amino acids, including, Val, Leu, Ile, Met, Pro, Phe, and Ala; and u=uncharged polar amino acids, including, Thr, Ser, Tyr, Gly, Gln, and Asn.

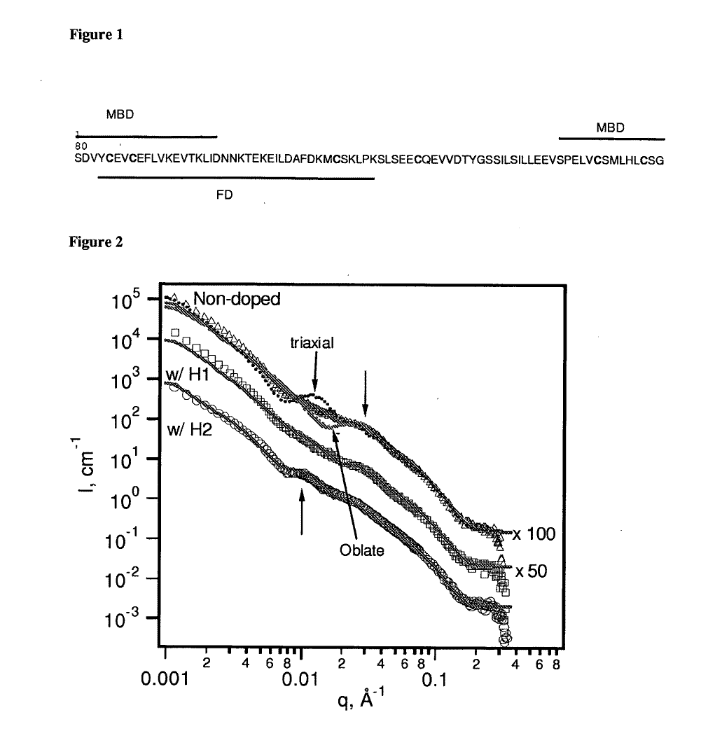

The functional domains of human SapC are shown in FIG. 1. The six cysteines (bold faced) and the N-glycosylation consensus sequence (*) are indicated. The two helical domains, H1 (YCEVCEFLVKEVTKLID) and H2 (EKEILDAFDKMCSKLPK) are labeled accordingly. The abbreviations MBD and FD stand for membrane binding and fusogenic domain, respectively. A fusogenic domain is located in the amino-terminal half of the SapC molecule, which includes the H1 and H2 peptides. The effects of SapC and its two helical domain peptides, H1 and H2, on the destabilization and restructuring of lipid membranes have been examined with atomic force microscopy (AFM). AFM indicates that SapC can destabilize and restructure the acidic membrane to form thicker “patches” on the surface, eventually leading to the dissolution of the bilayer. Membrane destabilization, as a result of SapC, also begins at defects, suggesting that the high curvature defects promote membrane destabilization. In contrast, neither H1 nor H2 alone have significant influence on membrane structure. H2 tends to interact with lipids where membrane defects are present, and then aggregates into rod-like structures. While AFM results show the influence of SapC and its H1 and H2 segments on supported membranes, the potential influence of these proteins on vesicle stability and morphology have not been understood. Here, SANS is used to characterize vesicles in the absence and presence of SapC, H1 and H2. This technique reveals both mesoscopic structural information related to vesicle size, shape and polydispersity, and nanoscopic information related to membrane thickness.

### Phospholipid Membrane and Formation of Liposomes

Liposomes are microscopic vesicles consisting of concentric lipid bilayers and, as used herein, refer to small vesicles composed of amphipathic lipids arranged in spherical bilayers. Structurally, liposomes range in size and shape from long tubes to spheres, with dimensions from a few hundred angstroms to fractions of a millimeter. Regardless of the overall shape, the bilayers are generally organized as closed concentric lamellae, with an aqueous layer separating each lamella from its neighbor. Vesicle size normally falls in a range of between about 20 and about 30,000 nm in diameter. Specific delivery of liposomes to a target tissue such as a proliferating cell mass, neoplastic tissue, inflammatory tissue, inflamed tissue, and infected tissue can be achieved by selecting a liposome size appropriate for delivering a therapeutic agent to said target tissue. For example, liposomes with a mean diameter of 180 nm may not accumulate in a solid tumor; liposomes with a mean diameter of 140 nm accumulate in the periphery of the same solid tumor, and liposomes with a mean diameter of 110 nm accumulate in the peripheral and central portions of that solid tumor.

Generally, liposomes are formed by subjecting a mixture of lipids to a mechanical force. For example, a wide variety of methods are currently used in the preparation of liposome compositions, such as, for example, solvent dialysis, French press, extrusion (with or without freeze-thaw), reverse phase evaporation, simple freeze-thaw, sonication, chelate dialysis, homogenization, solvent infusion, microemulsification, spontaneous formation, solvent vaporization, solvent dialysis, French pressure cell technique, controlled detergent dialysis, and others. See, e.g., Madden et al., Chemistry and Physics of Lipids, 1990. Liposomes may also be formed by various processes which involve shaking or vortexing. However, the ability to provide a method and composition whereby liposomes may be spontaneously formed without the need for application of a mechanical force is beneficial in that additional equipment and steps are not required, thereby reducing time and cost associated with their preparation. The present invention addresses this need.

Low polydispersity, spontaneously forming unilamellar vesicles (ULV) can be found in the phase diagram of ternary phospholipid mixtures containing long- and short-acyl chains. (see, for example, Nieh, M.-P.; Harroun, T. A.; Raghunathan; V. A., Glinka; C. J.; J. Katsaras Biophys. J. 2004, 86, 2615-2629; Egelhaaf, S. U.; Schurtenberger, P. Phys. Rev. Lett. 1999, 82, 2804-2807; Nieh, M.-P.; Raghunathan, V. A.; Kline, S. R.; Harroun, T. A.; Huang, C.-Y.; Pencer, J.; Katsaras, J. Langmuir 2005, 21, 6656-6661. The ULV are formed either by increasing the temperature (see Lesieur, P.; Kiselev, M. A.; Barsukov, L. I.; Lombardo, D. J. Appl. Cryst. 2000, 33, 623-627; Nieh, M.-P.; Harroun, T. A.; Raghunathan, V. A.; Glinka, C. J.; Katsaras, J. Phys. Rev. Lett. 2003, 91, 158105) or diluting bilayered discoidal micelles (see V. A., Glinka; C. J.; J. Katsaras Biophys. J. 2004, 86, 2615-2629) where the short-chain lipid sequesters into the high curvature rim of the bilayered disk, minimizing its edge energy. The discoidal bilayered micelle-to-ULV transition takes place at a temperature corresponding to the long-chain lipid's gel-liquid crystalline (main transition temperature), implying that increased miscibility levels between the long- and short-chain lipids leads the short-chain lipid to diffuse from the disc's edge, causing the line tension to increase and the modulus of rigidity to decrease. This series of events give rise to the formation of monodisperse ULV. (see Fromherz, P. Chem. Phys. Lett. 1983, 94, 259-266) These ULV are stable over extended periods of time, namely weeks (see Nieh, M.-P.; Harroun, T. A.; Raghunathan, V. A.; Glinka, C. J.; Katsaras, J. Phys. Rev. Lett. 2003, 91, 158105), and are thus considered to be good candidates as drug carriers.

### Compositions and Methods for Spontaneous Formation of Liposomes

One method of forming liposomes involves the use of long- and short-chain lipids, wherein both lipid species are di-saturated zwitterionic phospholipids doped with small amounts of an acidic long-chain lipid such as dimyristoyl phosphatidylglycerol (DMPG). In the instant disclosure, the morphology of one embodiment of spontaneously forming liposomes, is described. In this embodiment, the mixture of lipds used to spontaneously form liposomes comprises dipalmitoyl and dihexanoyl phosphatidylcholine (DPPC and DHPC, respectively), and the acidic, long-chain lipid dioleoyl phosphatidylserine (DOPS), though it will be readily understood to one of skill in the art that various modifications and substitutions may be made to this combination to arrive at other suitable embodiments within the scope of the invention. The mixture further includes at least one prosaposin-derived protein or polypeptide or variant or analogue thereof.

The spontaneously formed liposomes may be made by carrying out the following steps: 1) providing a mixture of dry lipids and a prosaposin-derived protein or polypeptide; 2) adding an aqueous solution to the mixture; 3) allowing the mixture to form liposomes, wherein the liposomes are stable and do not require the addition of mechanical force to achieve their formation. In one embodiment, the lipids comprise at least one long-chain anionic lipid. In another embodiment, the lipids comprise at least one anionic long-chain lipid and at least one short chain lipid. The prosaposin-derived protein or polypeptide may be selected from the group consisting of Saposin C (SEQ ID No. 2), H1 (SEQ ID No.3), H2 (SEQ ID No.4), H3 (SEQ ID No.5), H4 (SEQ ID No.6), H5 (SEQ ID No.7), and mixtures thereof. Prosaposin is represented in SEQ ID No. 1. The prosaposin-derived proteins may further comprise analogues or derivatives of Saposin C, H1, H2, H3, H4, H5, and mixtures thereof.

The aqueous solution may be any physiologically compatible solution capable of solubilizing the lipids and prosaposin-derived protein or polypeptide such that a liposome spontaneously forms. Non-limiting examples of aqueous solutions include, for example, water, deionized water, saline, and phosphate buffered saline (PBS). The molar concentration of total protein and lipid upon addition of the aqueous solution is about 300 uM or about 400 uM or about 500 uM or up to 1000 uM. The pH of the aqueous solution is about 7.4 or about 7.0-7.6, or about 6.8 to about 7.8.

After addition of the aqueous solution, the lipid and protein mixture spontaneously form liposomes. It will be understood to one of ordinary skill in the art, however, that the mixture may be vortexed or mixed to speed or otherwise facilitate formation of the liposomes.

In an alternative embodiment, the method may comprise the steps of providing one or more dry phospholipids and a prosaposin-derived protein or polypeptide; adding an organic solvent for form a first mixture; freeze-drying the first mixture; adding an aqueous solution to the first mixture to form a second mixture; allowing the second mixture to form liposomes. The one or more dry phospholipids may comprise at least one anionic long-chain phospholipid and/or at least one short chain phospholipid. The prosaposin-derived protein or polypeptide may be Saposin C or a fragment such as H1, H2, H3, H4 or H5. In this embodiment, the organic solvent may be any suitable organic solvent, for example, t-butanol (preferred), isopropanol, 1-propanol, ethanol, ethyl ether, methanol, or DMSO. The organic solvent comprises about 80-90% or about 70-95% or about 50-95% of the final first mixture prior to freeze drying. The first mixture may be stored for months or years prior to the addition of an aqueous solution used to form the liposomes.

The use of a negatively charged long-chain lipid such as DOPS instead of zwitterionic lipids only is believed to optimize the interactions between Saposin C or its fragments such as the H1 and H2 peptides and the acidic lipid aggregates, and is uniquely suited for the spontaneous formation of liposomes, providing a novel and useful means for forming liposomes for treatment of disease. In alternative embodiments, the negatively charged long-chain lipid may be selected from dioleoyl phosphatidylserine (DOPS), Dioleoylphosphatidyl-glycerol (DOPG), 1,2-dioleoyl-phosphatidyinositol (DOPI) and 1,2-dioleoylphosphatidic acid (DOPA) or combinations thereof. The negative long chain lipids of the present invention may be any long chain phospholipid that has a carbon chain about 14 to about 24 carbons in length, or about 18 to about 20 carbons in length. An exhaustive list of lipids is available at www.avantilipids.com. One skilled in the art will appreciate which lipids can be used in the present invention. While any combination of long and short chain lipids may be used, some combinations yield more stable liposomes. For example, while not intending to limit the present invention, the following may guide selection of the composition from which liposomes are formed: where long-chains of about 20 to about 24 carbons in length are used, short-chain lipids having lengths of about 6 to about 8 may be used for improved liposome stability. Where long-chain lengths of about 14 to about 18 are used, short-chain lipids having lengths of about 6 to about 7 may be used for improved liposome stability. While these combinations of lipids yield more stable liposomes, other combinations may successfully be used, and are not intended to be disclaimed.

The potential for stable ULV formation is further maximized through the addition of short and long-chain lipids as described herein. The short-chain lipid may be any suitable short chain lipid as understood by one of ordinary skill in the art. In one embodiment, for example, the short chain lipid is a neutral short chain phosphatidylcholine lipid such as dipalmitoyl phosphatidylcholine (DPPC). The short-chain lipid may also be a short-chain phosphatidylserine lipid such as DHPS. With the addition of the short chain lipid, as described herein, the liposome population formed is generally monodisperse. Without the short-chain lipid, the population is polydisperse, having variance in the size and shape of the resulting liposomes. As a result of adding the short-chain phospholipids, it is possible to achieve a monodisperse population, improving the ability to control the pharmacokinetics and bioavailability of the resulting preparation.

In one particular embodiment, the lipid mixture used to synthesize saposin-C liposomes comprises the negatively charged lipid dioleoylphosphatidyl-serine (DOPS) wherein a small amount of the neutral long chain lipid dipalmitoyl phosphatidylcholine (DPPC) and the neutral short-chain lipid dihexanoyl phosphatidycholine (DHPC) is added. In this particular embodiment, the [DOPS]:[DPPC] molar ratio ranges from 1:1 to 10:1 with ([DPPC]+[DOPS])/[DHPC] equal to about 4. In an alternative embodiment, DHPC is substituted or combined with DHPS.

Any lipid known in the art corresponding in charge and length may be used. Samples containing this composition of lipids doped with small amount of saposin C do not destabilize, but large aggregates can precipitate out of the solution for the system with a higher concentration of saposin C, indicating destabilization of the membrane. The DOPS/DPPC/DHPC samples are stable over a period of 24 months, indicating that the addition of the neutral long chain lipids and short chain lipids enhance the stability of the aggregates. However, any combination of long and short chain lipids may be used in accordance with the invention as described herein. Table 1 illustrates non-limiting examples of long chain and short chain lipids that may be used in carrying out the methods of the instant invention.

TABLE 1
Combinations of Long Chain and Short Chain Phospholipids

| | |
|---|---|
| Long-Chain Phospholipid | Short-Chain Phospholipid |

| | | |
|---|---|---|
| 18:1 PS | 18:0 PC | 06:0 PC (DHPC) |
| 18:1 PS | | 06:0 PC (DHPC) |
| 18:1 PS | 18:0 PC | 06:0 PS (DHPS) |
| 18:1 PS | | 06:0 PS (DHPS) |
| 18:2 PS | 18:1 PG | 06:0 PS (DHPS) |
| 18:0-18:1 PS | 18:1 PE | 06:0 PC(DHPC) |
| 16:0 PS | 16:1 PC | 05:0 PC |
| 20:4 PS | 20:1 PC | 07:0 PC |

Further, the presence or absence of saturating hydrocarbons on the lipid chain effect liposome stability. For example, lipids having chain lengths of about 18 or greater are used, the phospholipid may be saturated or unsaturated, preferably unsaturated. For shorter long-chain lipids such as those having about 14 to about 16 carbons, the lipid may be unsaturated, but use of saturated lipids yields improved performance of the present invention.

Non-limiting examples of lipid ratios are as follows. The molar ratio of the selected neutral phospholipid to the selected negative phospholipid in the composition is about 1 to 10 (about 10% neutral phospholipids), or about 1 to 5 (about 20% neutral phospholipids), or about 1 to 1 (50% neutral phospholipids). The molar ratio of the selected long-chain phospholipid to the selected short-chain lipid in the composition is about 4 to 1 (about 20% short-chain), and can be about 10 to 1 (10% short-chain) to about 3 to 1 (about 33% short-chain).

One example of the long-chain to short chain ratio in one embodiment is as follows: [neutral long-chain lipid]+[acidic long-chain lipid])/[neutral or anionic short-chain lipid] is about 4. As another example, in one embodiment, the molar ratio of DOPS to DPPC in the mixture ranges from about 10-8 to 1, or about 7-6 to 1, or about 5-3 to 1 or about 1-2 to 1, with ([DPPC]+[DOPS])/DHPC=about 4. In other embodiments, the [neutral long-chain lipid]+[acidic long-chain lipid])/[neutral or anionic short-chain lipid] may be about 2 to about 10 or about 3 to about 8 or about 4 to about 7. Appropriate lipids for use in the present invention may be selected from any lipids known in the art or as provided at www.avantilipids.com.

The liposomes of the present invention may further comprise one or more pharmaceutical agent and/or imaging agent that have been trapped in the aqueous interior or between bilayers, or by trapping hydrophobic molecules within the bilayer. Several techniques can be employed to use liposomes to target encapsulated drugs to selected host tissues, and away from sensitive tissues. These techniques include manipulating the size of the liposomes, their net surface charge, and their route of administration.

The liposomes of the present invention may also be delivered by a passive delivery route. Passive delivery of liposomes involves the use of various routes of administration, e.g., intravenous, subcutaneous, intramuscular and topical. Each route produces differences in localization of the liposomes.

The liposomes of the present invention are also ideal for delivery of therapeutic or imaging agents across the blood-brain barrier. The present invention relates to a method by which liposomes containing therapeutic agents can be used to deliver these agents to the CNS wherein the agent is contained within a liposome comprised of the above referenced lipids and saposin C, prosaposin or a variant of saposin. The liposome containing a therapeutic agent can be administered via IV injection, IM injection, trans-nasal delivery, or any other transvascular drug delivery method, using generally accepted methods in the art.

Without intending to be limited by theory, one possible mechanism as to how saposin-mediated membrane fusion occurs is through protein conformational changes. Of the pro-saposin derived proteins, saposin A and saposin C show the highest degree of amino acid identity/similarity. Computationally, both proteins are predicted to fold into amphipathic helical bundle motifs. In general, the saposin-fold is a common super secondary structure with five amphipathic α-helices folded into a single globular domain and is common to both proteins. In one embodiment, the folding is along a centrally located helix at amino-terminal, against which helices 2 and 3 are packed from one side and helices 4 and 5 from the other side. This fold may provide an interface for membrane interaction.

A mechanism for saposin-mediated membrane fusion with anionic phospholipid membranes is thought to be a two-step process. In the first step, electrostatic interactions between the positively charged amino acids (basic form), lysine (Lys) and arginine (Arg), of the saposins and the negatively charged phospholipid membrane results in an association between these two species (see FIG. 1). In the second step, intramolecular hydrophobic interactions between the helices of two adjacent saposin proteins brings the two membranes in close enough proximity for fusion of the membranes to take place (see FIG. 2).

Thus, in accordance with the present invention, the association of saposins, and in particular saposin C, with a lipid generally requires a pH range from about 5.5 or less since the initial association of saposin C with the membrane arises through an electrostatic interaction of the positively charged basic amino acid residues of saposin C with the anionic membrane. Thus, it is highly desirable to have the basic amino acids exist in their protonated forms in order to achieve a high number of electrostatic interactions. This can be accomplished, for example, by addition of an acidic solution to the prosaposin-derived protein or polypeptide prior to combining the protein or polypeptide with the lipid mixture.

Alternatively, related fusion proteins and peptides derived from the saposin family of proteins may not have this lower pH range limitation and thus the pH range of other membrane fusion proteins and peptides can range from physiological pH (pH of about 7) to lower pH ranges.

### Structural Analysis of DOPS/DPPC/DHPC Liposomes

DOPS (a negatively charged long-chain lipid), DPPC (a neutral long-chain lipid) and DHPC (a neutral, short-chain lipid) in powder form (available from Avanti Polar Lipids, Alabaster, Ala.) and is used without additional purification. For DLS measurements, the [DOPS]:[DPPC] molar ratio ranges from 1:1 to 10:1 with ([DPPC]+[DOPS])/[DHPC]=4 for all samples. Dry lipid mixtures are dissolved in filtered, ultra-pure H2O (Millipore EASYpure UV) at a total lipid concentration of 10 wt. % and mixed by vortexing and temperature cycling, between 4 and 50° C.

For preparation of liposomes containing protein, SapC was over expressed in E. coli cells using the IPTG-inducing pET system, and His-tag proteins were eluted from nickel columns. After dialysis, the proteins were further purified by HPLC chromatography as follows: The C4 reverse phase column was equilibrated with 0.1% trifluoroacetic acid (TFA) for 10 minutes, then the proteins were eluted in a linear (0-100%) gradient of 0.1% TFA in acetonitrile over 60 minutes. The major protein peak was collected and lyophilized, while protein concentrations were determined as described previously. H1 (YCEVCEFLVKEVTKLID) and H2 (EKEILDAF DKMCSKLPK) peptides were synthesized by SynPep Corp. (California, USA) and dissolved in D₂O at a concentration of 1.5 mg/mL. The 0.1 wt. % lipid solution with [DOPS]/[DPPC]=10 and ([DPPC]+[DOPS]/DHPC=4 was then added to the two peptide solutions (1.5 mg/mL) at a volume ratio of 12:1, and the SapC solution with a volume ratio of 1:1, yielding a final peptide (or SapC) concentration of 62.5 μM, (molar ratio of [lipid]/[peptide]=22/1) a concentration higher than the SapC required to induce membrane destabilization. A sample containing only 6.25 μM of SapC ([lipid]/[SapC]=220/1) was also prepared for comparison purposes.

In developing SapC-ULV complexes, the effect of the composition on the DOPS/DPPC/DHPC ULV system was characterized, a system suitable for the study of SapC-membrane interactions. In particular, the aggregate morphologies of this lipid mixture was determined via dynamic light scattering (DLS), transmission electron microscopy (TEM) and SANS measurements. SANS was then used to characterize the influence of H1, H2 and SapC on these ULV.

For aggregates in the absence of peptides, DLS and TEM results confirm the presence of a bimodal population of ULV with diameters of the order of ˜200 and >500 nm, consistent with fits to SANS data using a combination of form factors for oblate ellipsoidal vesicles, and triaxial ellipsoidal shells. SANS data reveal that SapC promotes aggregation of ULV at high concentrations (62.5 μM) while at lower concentrations (6.25 μM), the ULV structure is unperturbed. Both H1 and H2 induce small decreases to the membrane thickness. While the H1 peptide does not appear to modify ULV size or their size distribution, H2 shifts the equilibrium between the two ULV aggregates present. Qualitatively, these results are consistent with the previous AFM findings.

To determine the structure and stability of the resulting liposomes, the homogenized 10 wt. % solutions are first progressively diluted into 5, 2, 1, 0.5 and 0.1 wt. % samples. Prior to DLS measurements, the 0.1 wt. % stock lipid samples are diluted 5, 50 and 200 fold, and are analyzed using an N4⁺ particle sizer (Coulter, Miami, Fla.). Using this method, it was determined that diluting the system had no effect on the size of the particles. For SANS experiments, the same sample preparation protocol was applied to the [DOPS]/[DPPC]=10 sample except that D₂O (99.9%, Chalk River Laboratories, Chalk River, ON), instead of H₂O, was used to obtain a sample with a total lipid concentration of 0.5 wt. %. The 0.5 wt. % sample was then further diluted into 0.1 and 0.05 wt. % mixtures using an acidic buffer composed of equal-volumes of 0.1N sodium acetate (NaAc) and 0.1N acetic acid (HAc). The resultant solution had a pH value of 4.78±0.02 in D₂O and was stable over a 12-fold dilution with D₂O.

SANS experiments were carried out on the 30 m NG7 SANS instrument located at the National Institute of Standards and Technology (NIST) Center for Neutron Research (NCNR, Gaithersburg, Md., USA). 8.09 Å wavelength (λ) neutrons, a neutron focusing lens and a long sample-to-detector distance (SDD=15.3 m) were used to reach the lowest scattering vectors [q=4π/λ·sin(θ/2), where θ is the scattering angle]. Two other SDD (5 and 1 m) were also used, covering a combined q range from 0.002 to 0.35 Å⁻¹ for all three SDD. Raw 2-D data were corrected for detector sensitivity, background, empty cell scattering and sample transmission. The corrected data were then circularly averaged, around the beam center, yielding the customary 1-D data. These data were then put on an absolute intensity scale using the known incident beam flux. The incoherent plateau was determined by averaging the intensity of 10-20 high q data points and then subtracted from the reduced data.

ULV size was determined by photon correlation spectroscopy^{23,24} using an N4+ sub-micron particle size analyzer (Coulter, Miami, Fla.). Large vesicles were found to be polydisperse with diameters between 20-800 nm. The data were acquired at an angle of 90° and analyzed using a size distribution process (SDP) with an autocorrelation function. ANOVA analysis was used to determine statistical significance and error bars denote the standard deviation.

TEM images were taken with a Hitachi TEM (H-7600, HITACHI, Japan) operated at an acceleration voltage of 80 kV. A droplet of each sample was placed on a nickel grid coated with a support formvar film (200 mesh, a thickness range from 30 to 75 nm, Electron Microscopy Sciences, PA). The grid was placed on filter paper at room temperature for 2 h prior to TEM analysis. Background was optimized at high magnification, while the area of interest was located at low magnification (50-1,000×). A single vesicle could be viewed up to 50,000× magnification. TEM micrographs were taken using a dual AMT CCD digital camera (2K×2K, 16 bit) with appropriate image acquisition software.

Because SapC only functions in the presence of negatively charged unsaturated lipids (i.e., DOPS) at acidic conditions,²³ the present system is composed mainly of DOPS and lesser amounts of DPPC and DHPC. DLS results of the various [DOPS]/[DPPC] molar ratio samples (Table 1) indicate that only the [DOPS]/[DPPC]=10 sample exhibits a bimodal size distribution, while the remaining samples contain at least three populations. For this reason, only the [DOPS]/[DPPC]=10 sample was used to further investigate the effects of SapC, H1 and H2 on the structure of these ULV. DLS data also show that the structures of the DOPS/DPPC/DHPC samples are stable over a period of 12 months (Table 2). This is evidence that the addition of DPPC and DHPC dramatically enhances, compared to sonicated DOPS, the stability of these aggregates, and offer the possibility that they can be used in practical applications.

Note that the apparent sizes, or hydrodynamic radii of ULV as calculated from DLS results are based on the assumption that the ULV are spherical. As discussed in detail in literature,²⁹ for prolate or oblate vesicles, an accurate determination of the vesicle axial ratio requires measurement of both the vesicle hydrodynamic radius and radius of gyration, by DLS and static light scattering (SLS), respectively. For ellipsoidal vesicles, the apparent hydrodynamic radius will lead to a small (<10%) underestimate or overestimate of in ULV mass or surface area, for oblate or prolate vesicles, respectively.

TABLE 2
Hydrodynamic radii (rim) from DLS data of DOPS/DPPC/DHPC
aggregates in solution, where ([DOPS] + [DPPC])/DHPC =
4. The bracket indicates the population percentage of each aggregate.

| DOPS/DPPC | Duration | R_{H}, nm (%) | | |
|---|---|---|---|---|
| Ratio | (Days) | 1-100 | 100-200 | 400-800 |
| 1 | 1 | 40 (79) | 145 (12) | 441 (9) |
| 1 | 40 | 42 (76) | 173 (7) | 705 (17) |
| 5 | 1 | 29 (78) | 157 (11) | 570 (11) |
| 5 | 40 | None | 147 (51) | 689 (49) |
| 10 | 1 | None | 138 (70) | 582 (30) |
| 10 | 40 | None | 178 (56) | 746 (44) |
| 10 | 240 | None | 161 (49) | 452 (51) |
| 10 | 365 | None | 159 (51) | 471 (49) |

FIG. 2 shows SANS data of the 0.1 wt. % lipid mixture ([DOPS]/[DPPC]=10) and the H1- and H2-doped lipid mixtures in acetate D₂O buffer. Lower levels of SapC (6.25 μM) do not appear to perturb the size of ULV or their membrane structure (the same as non-doped mixtures). In the case of high SapC levels (62.5 μM), the SapC-doped lipid system forms large aggregates that precipitate out of solution (not amenable for SANS), an observation consistent with previous studies indicating that SapC destabilizes membranes.²³ Therefore, we only focus on the effects of H1 and H2 on the structure of the vesicles. SANS data of 0.1% DOPS/DPPC/DHPC (triangles), H1-doped (squares) and H2-doped (circles) mixtures is shown in FIG. 2. Solid lines represent best-fits to the data. The two broad peaks, indicated by arrows, are the result of correlation lengths present in the system. The dots and dashes lines are, respectively, the results of the tri-axial ellipsoidal and oblate shell models used to fit the 0.1% non-doped system. The scattering curves of the non SapC-doped samples shown in FIG. 2 share a common feature in that they contain two broad peaks centered along q ˜0.01 and 0.03 Å⁻¹, indicative of the presence of two correlation lengths in the system. To better understand the origins of these peaks, a 0.05 wt. % pure lipid mixture was also examined. SANS data of the 0.05 wt. % sample can be scaled to overlap the 0.1 wt. % sample data indicating that the two broad peaks are inherent to the aggregate morphologies and not the result of interparticle interactions.

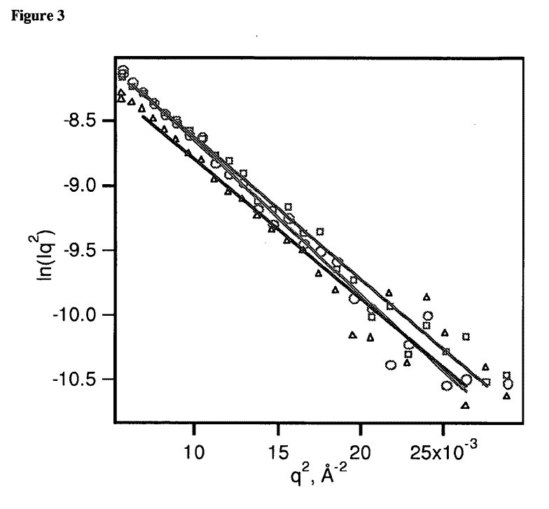

For q>0.06 Å⁻¹ the differences in intensity among the three data sets (FIG. 2) are indistinguishable. Analysis of a modified Guinier plot³⁰ (also known as Kratky-Porod plot) applied to all three samples, where ln(I·q²) has a linear relationship with q² in the range between 5×10⁻³ and 2.5×10⁻² Å⁻², indicate the existence of a lamellar structure. FIG. 3 shows the modified Guinier plot for the 0.1 wt. % DOPS/DPPC/DHPC mixture (circles), H1-doped system (triangles) and H2-doped (squares) systems. The lines represent the best-fits to the data. The bilayer thickness is then derived from the square root of the slope multiplying by \sqrt{12}. The best-fit results show that the non-doped mixture forms the thickest bilayer (37.7±0.7) Å, while the H1- and H2-doped bilayers are slightly thinner (35.8±0.8 and 36.2±0.6 Å, respectively).

For q≦0.06 Å⁻¹, the scattering curve of the H1-doped system, compared to the H2-doped system, is similar to that of the non-doped lipid mixtures, implying that no significant change is taking place in the aggregate morphology upon doping the membranes with H1 peptide. This observation is also consistent with a previous AFM report, where H1 was found to have no effect on 1-palmitoyl-2-oleoyl phosphatidylserine (POPS) bilayers. In the case of SANS data of the H2-doped sample, qualitative differences from that of the pure lipid mixture are observed in the slope of the low-q region (q<0.003 Å⁻¹) data, and the width and intensities of the two peaks.

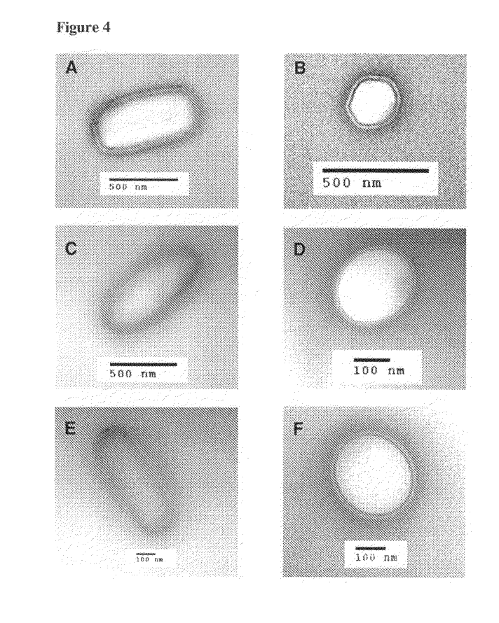

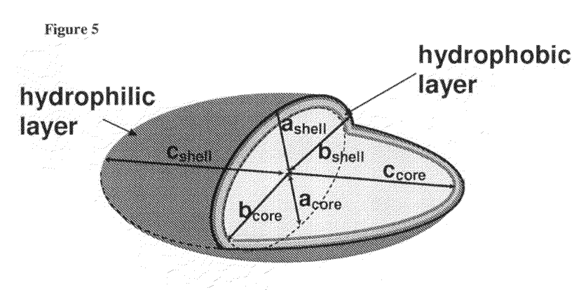

TEM images of all three samples were obtained and particles with two populations of morphologies were observed. FIG. 4 shows a representative TEM images of a DOPS/DPPC/DHPC mixture (A and B), and H1-doped (C and D), and H2-doped (E and F) mixtures. The tri-axial ellipsoidal vesicles, i.e., A, C and E, are all of similar size (projected cross-sectional area 150-200 nm×600-800 nm), as are the oblate vesicles with projected radii around 100-150 nm. These dimensions are consistent with the best-fit results of the SANS data, according to the mathematical model for a unilamellar tri-axial ellipsoidal vesicle as follows:

The model for a unilamellar tri-axial ellipsoidal vesicle is depicted as an ellipsoidal shell (FIG. 4) with different core lengths along the three prime axes, a_{core}, b_{core} and c_{core} (a_{core}≦b_{core}≦c_{core}). The shell lengths along the axes, aₛₕₑₗₗ, bₛₕₑₗₗ and cₛₕₑₗₗ, are defined as (a_{core}+l), (b_{core}+l) and (c_{core}+l), respectively, where l is the bilayer thickness. Note that this approximation does not assume a constant bilayer thickness over the entire ULV along the bilayer normal direction. The form factor for a tri-axial ellipsoidal shell averaged over all possible orientations, Pₜᵣᵢₐₓ(q), can thus be expressed as

$$\begin{matrix}{P}_{triax} ( q ) = \frac{1}{{V}_{triax}} {\int }_{- 1}^{1} {\int }_{- 1}^{1} {{A}_{triax} ( {a}_{core ,} {b}_{core ,} {c}_{core ,} l , x , y , q )}^{2} d x d y , & ( A \text{-} 1 ) \\ {A}_{triax} ( q ) = 3 ( {ρ}_{{D}_{2} O} - {ρ}_{lipid} ) [ \frac{{V}_{total} {j}_{1} ( {u}_{shell} )}{{u}_{shell}} - \frac{{V}_{core} {j}_{1} ( {u}_{core} )}{{u}_{core}} ] , & ( A \text{-} 2 )\end{matrix}$$

where j₁ is the first order spherical Bessel function

$${j}_{1} ( x ) = \frac{sin x}{{x}^{2}} - \frac{cos x}{x} ,$$

uᵢ is defined as

$$q \sqrt{{a}_{i}^{2} {cos}^{2} ( \frac{π x}{2} ) + {b}_{i}^{2} {sin}^{2} ( \frac{π x}{2} ) ( 1 - {y}^{2} ) + {c}_{i}^{2} {y}^{2}}$$

(i represent shell or core) and Vₜₒₜₐₗ and V_{core} are the total and core volumes of the ellipsoid, respectively. ρ_{D}_{₂}_{O} and ρ_{lipid} denote the scattering length densities of D₂O and lipid.

For oblate ellipsoidal vesicles, the form factor P_{oblate}(q) can be obtained by letting c_{core}=b_{core}. Thus, uᵢ becomes

$$q \sqrt{{a}_{i}^{2} {cos}^{2} ( \frac{π x}{2} ) + {b}_{i}^{2} {sin}^{2} ( \frac{π x}{2} )} .$$

Moreover, the Schultz function, f(a), is employed to describe the distribution of the shorter axis (i.e., a_{core}) as shown below

$$f ( a ) = \frac{{p}^{\frac{- 2}{{p}^{2}}}}{〈 a 〉 Γ ( \frac{1}{{p}^{2}} )} {( \frac{a}{〈 a 〉} )}^{\frac{( 1 - {p}^{2} )}{{p}^{2}}} exp ( - \frac{a}{{p}^{2} 〈 a 〉} ) , \qquad ( A \text{-} 3 )$$

where <a> is the average of a. p is the polydispersity defined as σ/<a>, where σ is the standard deviation of a. A reasonable value for p is in the range of 0-1.⁴⁵ The Gamma function, Γ(1/p²), is used to normalize the integral of the Schultz function. Eq. A-3 is put inside the inner integration of Eq. A-1 to be

$${P}_{oblate} ( q ) = \frac{1}{{V}_{oblate}} {\int }_{- 1}^{1} {\int }_{0}^{∞} f ( {a}_{core} ) \cdot {{A}_{oblate} ( {a}_{core} , {b}_{core} , l , x , q )}^{2} d {a}_{core} d x \qquad ( A \text{-} 4 )$$

The total scattering intensity for non interacting vesicles (oblate and tri-axial ellipsoid) can then be written as

I(q)=(Φₗᵢₚ−ΦD_{oblate})Pₜᵣᵢₐₓ(q)+Φ_{oblate}P_{oblate}(q), (A-5)

where Φₗᵢₚ and Φ_{oblate} are the volume fractions of the total lipid and oblate shell in solution, respectively. The fitting procedure is written in IGOR programming code, which is revised from the data analysis package developed by NIST SANS group.

The proposed model for a unilamellar, tri-axial ellipsoidal bilayered vesicle is shown in FIG. 5. The hydrophilic (head groups) and hydrophobic (hydrocarbon tails) regions are shown. In the case of oblate vesicles, b equals c.

One morphology has a circular 2-D projection with a radius ˜150-200 nm, while the other morphology has an elongated ellipsoidal projection with the long and short axes of dimensions between 600 and 800 nm, and 100 and 200 nm, respectively. This result is consistent with the DLS data; however, the bimodal distribution can be either a mixture of spherical and ellipsoidal vesicles or that of oblate and tri-axial ellipsoidal vesicles, depending on the thickness of the particles along the axis perpendicular to the projection. Since the former one (mixture of spherical and ellipsoidal vesicles) does not fit our SANS data, we formulate a model that includes the oblate and tri-axial ellipsoidal vesicular shells for fitting the SANS result. (See mathematical model for a unilamellar tri-axial bilayered vesicle, above). The oblate morphology has two equal-length major axes and one polydisperse minor axis (shown in FIGS. 4B, D, F), while the tri-axial ellipsoid morphology has three unequal length axes (FIGS. 4A, C, E). This model requires eight fitting parameters, namely, three axes for the tri-axial ellipsoidal shell, two axes for the oblate shell, polydispersity of the shorter axis for the oblate morphology, the bilayer thickness and the population ratio of triaxial-to-oblate. However, results from TEM and DLS measurements, as well as the Kratky-Porod analysis allows us to constrain the bilayer thickness and the lengths of the two major axes in the case of the oblate shell, and the two longer axes for the triaxial ellipsoidal shell. This leaves the lengths of the shorter axes, the polydispersity of the shorter axis of the oblate shell and the population ratio of triaxial-to-oblate as free fitting parameters. The best-fit result shows that the oblate and tri-axial ellipsoids have a bilayer thickness of 40±5 Å, slightly larger than the result obtained from the modified Guinier plot. The shortest axis of the tri-axial ellipsoid and minor axis of the oblate morphologies are 250±20 Å and 100±10 Å, respectively. These features give rise to the broad peaks (˜0.01 and ˜0.03 Å⁻¹) in the SANS data. Moreover, the best-fit data for the lengths of the major axis of the oblate (˜150 nm) and the two longer axes of the ellipsoid (˜200 and 500 nm) morphologies are consistent with the TEM result shown in FIG. 4. The percent population of oblate ellipsoids is found to be ˜40±10% in the case of the H2-doped mixture, while it is ˜60±10% for the non- and H1-doped mixtures. The fact that a higher intensity of the first peak (˜0.01 Å⁻¹) is observed in H2-doped system, indicative of higher population of tri-axial ellipsoidal vesicles, is consistent with the best-fit result. It therefore seems that the H2 peptide favors the formation of tri-axial ellipsoidal vesicles. In summary, all three techniques point to the presence of two morphologies, namely tri-axial and oblate ellipsoids.

The DPPC/DHPC/DOPS mixtures used to form ULV systems using SapC and SapC fragments (H1 and H2) surprisingly showed unexpected behavior of the lipid mixture. Based on our previous experimental results,³ our initial expectations were to find appropriate conditions for the formation of monodisperse, spherical ULV. However, as we noted above, we find that, while DPPC/DHPC/DOPS do form ULV, the size distributions of the mixtures examined by DLS were multimodal. In the case of bimodally distributed ULV, we find that each population has a non-spherical shape and a narrow size distribution. This observation leads us to speculate that the mechanism for present ULV formation is different from that taking place in DMPC/DHPC/DMPG mixtures.

In previous studies, we found that the formation of low-polydispersity ULV required heating the system from the low temperature bilayered micelle (bicelles) morphology. ULV size was found to be dependent on the size of the bicelles and was most likely modulated by such factors as, the rim line tension energy, the bilayer's bending rigidity and the rate of bicelle coalescence. Moreover, the DMPC/DHPC/DMPG bicelle→ULV transition was closely associated with the gel liquid crystalline transition of DMPC, which takes place at ˜23° C. If the DPPC/DHPC/DOPS were to exhibit a similar behavior, it is likely that the bicellar morphology would be found near or below −11° C., the temperature where DOPS' fatty acid chains undergo a melting transition. Since dilution of the DMPC/DHPC/DMPG mixtures, at high temperatures, led to the formation of ULV with a broad size distribution,³ we conclude that the ULV formation mechanism here is different from that previously investigated.

The prior art teaches that upon dilution and as a result of collective membrane fluctuations, pure DOPS lamellar stacks completely unbind forming polydisperse ULV, teaching away from the use of DOPS to form bimodal populations of liposomes. Thus, although pure DOPS suspensions can also form ULV, we can dismiss this as the mechanism of formation taking place here. We speculate that the non-spherical ULV observed are stabilized by having the neutral, short-chain DHPC populating the high curvature region of the ULV, while the long-chain DPPC provides the requisite rigidity needed for stable bilayers.

The present disclosure sets forth the unexpected finding that, although a bimodal ULV size distribution is observed, the polydispersities of the individual populations are reasonably low. It may be that these two populations represent equilibrium, minimum energy states which may exchange material freely, or it could be that the individual ULV are dynamic structures capable of switching back and forth between these two morphologies. The notion that morphologies freely transform has been predicted theoretically and may also be analogous to previous experimental observations where prolate free vesicles transformed into oblate vesicles, and vice versa.^{29,35,36}

The ellipsoidal ULV morphology is also unexpected, but could be a consequence of membrane lateral heterogeneities. It has recently been shown by SANS that ternary mixtures containing saturated and unsaturated lipids can exhibit lateral segregation.³⁷ Furthermore, it has been found that, as a function of increasing temperature through the gel→liquid crystalline transition, a complicated spherical-polygonal-ellipsoidal transition in giant ULV.³⁸ The seemingly polygonal shape (FIG. 4B) presumably resulted from the lateral phase separation between these two phases, where the DOPS and DHPC lipids are in the L_{α} phase, while DPPC is in gel phase. In addition, due to the different lipid species possessing different hydrocarbon chain lengths, each domain may contribute to determining the length of each of the ellipsoid's axes. To the best of our knowledge, monodisperse tri-axial ellipsoidal vesicles from pure phospholipid systems or lipid mixtures have not been previously reported, although there are examples of spherical vesicles transforming into oblate or irregular-shaped vesicles induced by the polymerization of actin. We speculate that the result could be due to the lateral phase separation within the membrane.

The best-fits to the high q data result in a bilayer thickness between 38 and 40 Å. Since the ellipsoidal shell model assumes a constant bilayer thickness along the prime axis, and a uniform scattering length density across the bilayer, the value for the bilayer thickness could be expected to be slightly greater than values obtained from more detailed models. The modified Guinier plot indicates that, compared to H1- and H2-doped ULV, non-doped ULV possess a thicker bilayer. This demonstrates that although H1 and H2 have a thinning effect on the bilayer, they do not destabilize the bilayer. Wang et al. have reported that H1 and H2 can inhibit SapC induced membrane fusion, implying that they possibly bind at the same DOPS site as SapC, thus reducing SapC's interaction with the membrane. Their results also showed that H1 is more effective than H2 in the inhibition of membrane fusion. This is consistent with the fact that H1 has a greater effect on bilayer thinning.

A previous AFM study has shown that H2 formed patches on the membrane, which were inferred to be rod-like structures populating regions of bilayer defects. SANS data indicate a population shift of particle morphologies from oblate to tri-axial ULV upon doping with H2, compared to non- and H1-doped ULV. Since the short-chain DHPC is known to create defects in the membrane, this lipid mixture may provide a suitable environment for H2 to associate with, leading to the preferred formation of triaxial ellipsoidal ULV.

Through the use of SANS, TEM and DLS, the various morphologies assumed by mixtures of DOPS/DPPC/DHPC were characterized. Two low-polydispersity morphologies are observed, namely oblate and tri-axial ellipsoidal ULV. Here, the ULV formation mechanism seems to differ from that reported previously and demonstrates that low-polydispersity ULV can be formed even in the absence of the long-chain lipid undergoing a gel→liquid crystalline transition. SANS result shows that H1 and H2 do not destabilize the bilayer morphology, a result consistent with previous AFM data, but that H1 has a greater effect on bilayer thinning. Moreover, the addition of the H2 peptide does increase the ratio of triaxial-to-oblate ellipsoidal vesicles, presumably due to a change in miscibilities of various lipid components. On the other hand, the addition of SapC destabilizes the membrane and results in the precipitation, from solution, of large aggregates.

Increasing evidence suggests that liposomal vesicles are effective carriers for a variety of therapeutics. However, the efficacy of a particular system in treating disease and its commercial viability are of prime importance. In the instant disclosure, it is demonstrated that phosphatidylserine containing ULV have been shown to form spontaneously, are highly stable and of low-polydispersity. The protocol described is suited for scaled-up industrial production of SapC-bound ULV, useful for the development of SapC-PS ULV complexes which can then be tested for the in vivo transport of therapeutic agents.

The long chain lipids of the present invention may be any long chain phospholipid that has a carbon chain about 14 to about 24 carbons in length, or about 18 to about 20 carbons in length. An exhaustive list of lipids is available at www.avantilipids.com. One skilled in the art will appreciate which lipids can be used in the present invention. While any combination of long and short chain lipids may be used, some combinations yield more stable liposomes. For example, while not intending to limit the present invention, the following may guide selection of the composition from which liposomes are formed: where long-chains of about 20 to about 24 carbons in length are used, short-chain lipids having lengths of about 6 to about 8 may be used for improved liposome stability. Where long-chain lengths of about 14 to about 18 are used, short-chain lipids having lengths of about 6 to about 7 may be used for improved liposome stability. While these combinations of lipids yield more stable liposomes, other combinations may successfully be used, and are not intended to be disclaimed. Table 2 illustrates examples of phospholipid combinations that may be used to generate more stable liposomes. These examples, however, are not meant to imply that other combinations of phospholipids may not be used with the present invention.

TABLE 2
Non-Limiting Examples of Combinations of Long and
Short-Chain Phospholipids.

| Long-Chain | Short-Chain |
|---|---|
| Phospholipid Length | Phospholipid Length |
| (Number of Carbons) | (Number of Carbons) |
| 14 to 24 | 4 to 8 |
| 16 to 22 | 5 to 7 |
| 18 to 20 | 6 to 7 |
| 20 to 24 | 7 to 8 |
| 14 to 18 | 4 to 6 |

Further, the presence or absence of saturating hydrocarbons on the lipid chain effect liposome stability. For example, lipids having chain lengths of about 18 or greater are used, the phospholipid may be saturated or unsaturated, preferably unsaturated. For shorter long-chain lipids such as those having about 14 to about 16 carbons, the lipid may be unsaturated, but use of saturated lipids yields improved performance of the present invention.

Additional Agents

It is also contemplated to be a part of the present invention to prepare microspheres using compositions of matter in addition to the biocompatible lipids and polymers described above, provided that the microspheres so prepared meet the stability and other criteria set forth herein.

Propylene glycol may be added to remove cloudiness by facilitating dispersion or dissolution of the lipid particles. The propylene glycol may also function as a thickening agent that improves microsphere formation and stabilization by increasing the surface tension on the microsphere membrane or skin. It is possible that the propylene glycol further functions as an additional layer that coats the membrane or skin of the microsphere, thus providing additional stabilization. As examples of such further basic or auxiliary stabilizing compounds, there are conventional surfactants which may be used, e.g., U.S. Pat. Nos. 4,684,479 and 5,215,680.

Additional auxiliary and basic stabilizing compounds include such agents as peanut oil, canola oil, olive oil, safflower oil, corn oil, or any other oil commonly known to be ingestible which is suitable for use as a stabilizing compound in accordance with the requirements and instructions set forth in the instant specification.

In addition, compounds used to make mixed micelle systems may be suitable for use as basic or auxiliary stabilizing compounds, and these include, but are not limited to: lauryltrimethylammonium bromide (dodecyl-), cetyltrimethylammonium bromide (hexadecyl-), myristyltrimethylammonium bromide (tetradecyl-), alkyldimethylbenzylammonium chloride (alkyl=C12,C14,C16,), benzyldimethyldodecylammonium bromide/chloride, benzyldimethyl hexadecylammonium bromide/chloride, benzyldimethyl tetradecylammonium bromide/chloride, cetyl-dimethylethylammonium bromide/chloride, or cetylpyridinium bromide/chloride.

It has been found that the liposomes used in the present invention may be controlled according to size, solubility and heat stability by choosing from among the various additional or auxiliary stabilizing agents described herein. These agents can affect these parameters of the microspheres not only by their physical interaction with the lipid coatings, but also by their ability to modify the viscosity and surface tension of the surface of the liposome. Accordingly, the liposomes used in the present invention may be favorably modified and further stabilized, for example, by the addition of one or more of a wide variety of (a) viscosity modifiers, including, but not limited to carbohydrates and their phosphorylated and sulfonated derivatives; and polyethers, preferably with molecular weight ranges between 400 and 100,000; di- and trihydroxy alkanes and their polymers, preferably with molecular weight ranges between 200 and 50,000; (b) emulsifying and/or solubilizing agents may also be used in conjunction with the lipids to achieve desired modifications and further stabilization; such agents include, but are not limited to, acacia, cholesterol, diethanolamine, glyceryl monostearate, lanolin alcohols, lecithin, mono- and di-glycerides, mono-ethanolamine, oleic acid, oleyl alcohol, poloxamer (e.g., poloxamer 188, poloxamer 184, and poloxamer 181), polyoxyethylene 50 stearate, polyoxyl 35 castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, trolamine, and emulsifying wax; (c) suspending and/or viscosity-increasing agents that may be used with the lipids include, but are not limited to, acacia, agar, alginic acid, aluminum mono-stearate, bentonite, magma, carbomer 934P, carboxymethylcellulose, calcium and sodium and sodium 12, carrageenan, cellulose, dextran, gelatin, guar gum, locust bean gum, veegum, hydroxyethyl cellulose, hydroxypropyl methylcellulose, magnesium-aluminum-silicate, methylcellulose, pectin, polyethylene oxide, povidone, propylene glycol alginate, silicon dioxide, sodium alginate, tragacanth, xanthum gum, alpha-d-gluconolactone, glycerol and mannitol; (d) synthetic suspending agents may also be utilized such as polyethyleneglycol (PEG), polyvinylpyrrolidone (PVP), polyvinylalcohol (PVA), polypropylene glycol, and polysorbate; and (e) tonicity raising agents may be included; such agents include but are not limited to sorbitol, propyleneglycol and glycerol.

The diluents which can be employed to create an aqueous environment include, but are not limited to water, either deionized or containing any number of dissolved salts, etc., which will not interfere with creation and maintenance of the stabilized microspheres or their use as MRI contrast agents; and normal saline and physiological saline.

The biocompatible polymers useful as stabilizing materials for preparing the gas and gaseous precursor filled vesicles may be of natural, semi-synthetic (modified natural) or synthetic origin. As used herein, the term polymer denotes a compound comprised of two or more repeating monomeric units, and preferably 10 or more repeating monomeric units. The phrase semi-synthetic polymer (or modified natural polymer), as employed herein, denotes a natural polymer that has been chemically modified in some fashion. Exemplary natural polymers suitable for use in the present invention include naturally occurring polysaccharides. Such polysaccharides include, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrin, dextrose, polydextrose, pustulan, chitin, agarose, keratan, chondroitan, dermatan, hyaluronic acid, alginic acid, xanthan gum, starch and various other natural homopolymer or heteropolymers, such as those containing one or more of the following aldoses, ketoses, acids or amines: erythrose, threose, ribose, arabinose, xylose, lyxose, allose, altrose, lucose, mannose, gulose, idose, galactose, talose, erytirulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Accordingly, suitable polymers include, for example, proteins, such as albumin. Exemplary semi-synthetic polymers include carboxymethylcellulose, hydroxymethylcellulose, hydroxypropylmethyl-cellulose, methylcellulose, and methoxycellulose. Exemplary synthetic polymers suitable for use in the present invention include polyethylenes (such as, for example, polyethylene glycol, polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbons, fluorinated carbons (such as, for example, polytetrafluoroethylene), and polymethylmethacrylate, and derivatives thereof. Methods for the preparation of vesicles which employ polymers as stabilizing compounds will be readily apparent to those skilled in the art, once armed with the present disclosure, when the present disclosure is coupled with information known in the art, such as that described and referred to in U.S. Pat. No. 5,205,290, the disclosures of which are hereby incorporated herein by reference, in their entirety.

Alternatively, one or more anti-bactericidal agents and/or preservatives may be included in the formulation of the compositions, such as sodium benzoate, quaternary ammonium salts, sodium azide, methyl paraben, propyl paraben, sorbic acid, ascorbylpalmitate, butylated hydroxyanisole, butylated hydroxytoluene, chlorobutanol, dehydroacetic acid, ethylenediamine, monothioglycerol, potassium benzoate, potassium metabisulfite, potassium sorbate, sodium bisulfite, sulfur dioxide, and organic mercurial salts. Such sterilization, which may also be achieved by other conventional means, such as by irradiation, will be necessary where the stabilized vesicles are used for imaging under invasive circumstances, e.g., intravascularly or intraperitonealy. The appropriate means of sterilization will be apparent to the artisan based on the present disclosure.

Disaccharides

In addition to the foregoing additional ingredients, disaccharides may be added to the dry lipid mixture prior to or in concert with the addition of the aqueous solution may be added to improve liposome stability. Non-limiting examples of suitable disaccharides include, but are not limited to trehalose, sucrose, maltose, lactose, melibiose, galactose, glucose, fructose, or lactose. In general, the disaccharide comprises about 50 to about 100 mg of sugar per 10 mg total protein, i.e, a ratio of about 1:10 protein to disaccharide.

Antimembrane Agents

In addition, other lipids known as anticancer (or “antimembrane”) agents may be used with the described compositions and methods. For example, Edelfosine (sn-ET-18-OCH₃ or 1-O-Octadecyl-2-O-methyl-sn-glycero-3-phosphorylcholine) Miltefosine (Hexadecylphosphocholine), or other phospholipids, such as lysophosphatides, cardiolipin, ceramides, sphingomyelin, sphingosines, cerebrosides, cholesterol, modified forms of these lipids, and combinations of any of the aforementioned lipids, may be added to the compositions described herein. Exemplary compositions may comprise SapC (100 μM)+DOPS (280 μM)+Edelfosine (20 μM); or SapC (100 μM)+DOPS (290 μM)+Edelfosine (10 μM). The amount of Edelfosine may rang from about 2 to about 50 μM; the amount of DOPS may range from about 250 μM to about 298 μM where SapC is approximately 100 μM. Another exemplary range includes molar ratio of SapC:DOPS:Edelfosine from 1:3:0.2 to 1:10:0.7. Where SapC is described, it should be understood that other prosaposin derived proteins or polypeptides as described herein may be substituted or used in combination.

Although this invention has been described in connection with its most preferred embodiment, additional embodiments are within the scope and spirit of the claimed invention. The preferred device of this invention is intended merely to illustrate the invention, and not limit the scope of the invention as it is defined in the claims that follow.

What is claimed is:

1.-30. (canceled)

31. A composition for forming a nanovesicle comprising
a) at least one long-chain phospholipid;
b) at least one short-chain phospholipid; and
c) a prosaposin-derived protein or polypeptide,
wherein the nanovesicle is spontaneously formed upon addition of an aqueous solution.

32. The composition of claim 31, wherein the long-chain phospholipid is selected from the group consisting of dioleoylphosphatidylserine (DOPS), dioleoylphosphatidylglycerol (DOPG), dioleoylphosphatidylinositol (DOPI), dioleoylphosphatidic acid (DOPA), or a mixture thereof.

33. The composition of claim 31, wherein the short-chain phospholipid is selected from the group consisting of a phosphatidylserine, a phosphatidylcholine, a phosphatidylglycerol, a phosphatidylinositol, a phosphatidic acid, a phosphatidylethanolamine, or a mixture thereof.

34. The composition of claim 31, wherein the nanovesicle has a size distribution of vesicles comprising monomodal unilamellar vesicles, bimodal unilamellar vesicles, or trimodal unilamellar vesicles, or a mixture thereof.

35. The composition of claim 31, wherein the nanovesicle comprises oblate and tri-axial ellipsoidal unilamellar vesicles.

36. The composition of claim 31, wherein the prosaposin-derived protein is selected from the group consisting of saposin C, H1, H2, H3, H4, H5, or a mixture thereof.

37. A composition for forming a nanovesicle comprising
a) a long-chain phospholipid, wherein the long-chain anionic phospholipid is DOPS,
b) short-chain phospholipids, wherein the short-chain phosphohoplids are DPPC and DHPC, and
c) a prosaposin-derived protein or polypeptide, wherein the protein or polypeptide is selected from the group consisting of saposin C, H1, H2, H3, H4, H5,
or a mixture thereof.

38. The composition of claim 37, wherein the ratio of DOPS to DPPC is within a range of about 2 to about 20.

39. The composition of claim 31, wherein the long-chain phospholipid is selected from the group consisting of anionic long-chain phospholipids and neutral long-chain phospholipids, and wherein the amounts of the anionic long-chain phospholipid, the neutral long-chain phospholipid, and the short-chain phospholipid are governed by the formula (([neutral long-chain phospholipid]+[anionic or neutral long-chain phospholipid])/(short-chain phospholipid)), and wherein the result of the formula is within the range of about 2 to about 10.

40. The composition of claim 31 further comprising a pharmaceutically active agent.

41. A method of making a nanovesicle population comprising the steps of
i) providing a composition comprising at least one long-chain phospholipid, at least one short-chain phospholipid, and at least one prosaposin-derived protein or polypeptide;
ii) providing an aqueous solution; and
iii) combining the composition with the aqueous solution to spontaneously form a population of nanovesicles,
wherein the population of nanovesicles have a size distribution of vesicles comprising monomodal unilamellar vesicles, bimodal unilamellar vesicles, and trimodal unilamellar vesicles, or a mixture thereof.

42. The method of claim 41, wherein the long-chain phospholipid is selected from the group consisting of dioleoylphosphatidylserine (DOPS), dioleoylphosphatidylglycerol (DOPG), dioleoylphosphatidylinositol (DOPI), dioleoylphosphatidic acid (DOPA), or a mixture thereof.

43. The method of claim 41, wherein the short-chain phospholipid is a phosphatidylserine or a phosphatidylcholine.

44. The method of claim 43, wherein the short-chain phospholipid is selected from the group consisting of DHPC, DHPS, or a mixture thereof.

45. The method of claim 41, wherein the population of nanovesicles comprises oblate and tri-axial ellipsoidal unilamellar vesicles.

46. The method of claim 41, wherein the prosaposin-derived protein or polypeptide is selected from the group consisting of saposin C, H1, H2, H3, H4, H5, or a mixture thereof.

47. The method of claim 41, wherein the composition comprises DOPS, DPPC, DHPC and Saposin C.

48. The method of claim 41, wherein the composition comprises DOPS, DPPC, DHPC and H1.

49. The method of claim 41, wherein the composition comprises DOPS, DPPC, DHPC and H2.

50. The method of claim 41, wherein the composition comprises DOPS, DPPC, DHPC and H5.

51. The method of claim 41, wherein the composition comprises DOPS, DPPC, DHPC, Saposin C, H1 and H2.

52. The method of claim 41, wherein the composition comprises DOPS, DPPC, DHPS, and Saposin C.

53. The method of claim 41, wherein the composition comprises DOPS, DPPC, DHPS, and H1.

54. The method of claim 41, wherein the composition comprises DOPS, DPPC, DHPS, and H2.

55. The method of claim 41, wherein the composition comprises DOPS, DPPC DHPS, and H5.

56. The method of claim 41, wherein the composition comprises DOPS, DPPC, DHPS, Saposin C, H1 and H2.

57. The method of claim 41, wherein the ratio of the long-chain phospholipid to the short-chain phospholipid equals about 2 to about 20.

58. The method of claim 41, wherein the long-chain phospholipid is selected from the group consisting of anionic long-chain phospholipids and neutral long-chain phospholipids, and wherein the amounts of the anionic long-chain phospholipid, the neutral long-chain phospholipid, and the short-chain phospholipid are governed by the formula (([neutral long-chain phospholipid]+[anionic long-chain phospholipid])/(short-chain phospholipid)), and wherein the result of the formula equals about 2 to about 10.

59. The method of claim 58, wherein the short chain lipid is anionic or neutral.

60. The method of claim 41, wherein the nanovesicle further comprises a pharmaceutically active agent.
